(19) United States
(12) Patent Application Publication
(10) Pub. No.: US 2025/0345101 A1
(43) Pub. Date: Nov. 13, 2025

(54) Orthopedic Implant Kit

(71) Applicant: Neo Medical SA, La Villette (CH)

(72) Inventor: Morten Beyer, Rødkærsbro (DK)

(21) Appl. No.: 19/276,454

(22) Filed: Jul. 22, 2025

Related U.S. Application Data
(62) Division of application No. 17/105,683, filed on Nov. 27, 2020, now Pat. No. 12,396,760.
Application No. 17/105,683 is a division of application No. 15/939,338, filed on Mar. 29, 2018, now Pat. No. 10,888,356.
Application No. 15/939,338 is a continuation of application No. 14/890,631, filed on Nov. 12, 2015, now Pat. No. 10,058,355.

(30) Foreign Application Priority Data
May 13, 2013 (WO) PCT/IB2013/053892

(51) Int. Cl. A61B 17/70; A61B 17/88

(52) U.S. Cl. CPC A61B 17/7037; A61B 17/7002; A61B 17/7032; A61B 17/7038; A61B 17/708; A61B 17/7085; A61B 17/7086; A61B 17/7091; A61B 17/8875

(57) ABSTRACT

An orthopedic implant kit comprising a lockable poly-axial screw, a tissue dilatation sleeve, a screw driver, a screw extender, a rod, rod-reduction device, a set screw driver, a torque limiting device and a screw releasing device.

### CROSS REFERENCE TO RELATED APPLICATIONS

This application is a Divisional of application Ser. No. 17/105,683, filed on Nov. 27, 2020, which is a Divisional of application Ser. No. 15/939,338, filed on Mar. 29, 2018, which is a Continuation of application Ser. No. 14/890,631 filed on Nov. 12, 2015, which is the National Phase under 35 U.S.C. § 371 of International Application No. PCT/IB2014/060979, filed on Apr. 24, 2014, which claims the benefit under 35 U.S.C. § 119(a) to International Application No. PCT/IB2013/053892, filed in the International Bureau of the World Intellectual Property Organization (WIPO) on May 13, 2013, all of which are hereby expressly incorporated by reference into the present application.

### FIELD OF INVENTION

The present invention relates to orthopedics and more precisely to orthopedic items such as pedicle screws, rods and spine cages. The invention also relates to instruments which are used for manipulating those items.

### BACKGROUND

US 2013/0012999 discloses an orthopedic implant kit comprising several items, in particular a pedicle screw fixed to a mounting tube made of two half-shells which can be easily disassembled. Pedicle screws of the prior art can be divided in two main groups:
Mono-axial screws: The direction of the screw main axis is fixed with respect the screw head;
Poly-axial screws: The orientation of the screw main axis can be freely modified with respect to the screw head.

When implanting a pedicle screw into a bone several steps are needed. For almost each of those steps a dedicated instrument is used.

### GENERAL DESCRIPTION OF THE INVENTION

An objective of the present invention is to reduce the number of items which are required for manipulating and fixing an orthopedic implant (pedicle screw, nut, rod, etc . . . ).

Another objective is to reduce the number of instruments for manipulating those items.

Another objective is to facilitate the handling of the instruments.

Those objectives are met with the implant kit and the related items and instruments which are defined in the claims.

In a first embodiment the invention consists in an orthopedic implant kit comprising a lockable poly-axial screw, a tissue dilatation sleeve, a screw driver, a screw extender, a rod, rod-reduction means, a set screw driver, a torque limiting mechanism and a screw releasing instrument.

The lockable poly-axial orthopedic screw according to the invention comprises a head and a threaded portion which form two separate elements, fixed to each other but each element may be independently oriented along a specific direction. The threaded portion may, for instance, rotates around the screw head and may adopt several possible orientations. More precisely, the threaded portion may be oriented anywhere within a conical volume, the top of the cone corresponding to the contact point between the head and the threaded portion.

The screw furthermore comprises a locking element which, when activated, suppresses the relative movement between the threaded portion and the head. This configuration is named “mono-axial” because the threaded portion may be oriented along a single (fixed) axis with respect to the head. According one embodiment the locking element is a clip having a U-shape.

In this case the head and the threaded portion contains cavities which are adapted to receive the branches of the U-shape clip.

Preferably, in the mono-axial mode the head may still freely rotate around its own axis, with respect to the threaded portion. Such a mechanism may be obtained with a U-shape clip and with an annular groove located around the upper part of the threaded portion. In this case the branches of the clip are sliding within the annular groove.

In another embodiment the screw head contains at least one longitudinal relief, such as a groove or a ridge, which is dimensioned in a way as to receive a corresponding relief, such as a ridge or a groove, which is located within the distal end of a screw extender.

In another embodiment the screw comprises a concave seat located in the proximal end of the threaded portion and a corresponding convex shape located at the distal part of the screw head. This configuration reduces the screw length and increases the strength and the rigidity of the system.

The screw extender according to the invention comprises a hollow cylindrical body made of two half tubes separated by two opposite longitudinal slots having an open end towards the cylindrical body distal part, this later one being dimensioned to receive and hold a screw head. The cylindrical body furthermore comprises an internal threaded part.

According to one embodiment the cylindrical body is made of a single piece and the distal part is radially expandable by its own elasticity, in such a way as to allow an easy clipping and subsequent releasing of a screw head.

To facilitate its radial expansion, the screw extender may include expanding means, for instance an internal rotatable tube which, when rotated pushes away the two half tubes from each other.

In a preferred embodiment the internal part of the cylindrical body distal end contains at least one relief, such as a ridge or a groove, which is dimensioned to be received within the longitudinal relief of a screw head which includes a corresponding relief, as mentioned previously. With this configuration it hinders the distal part of the half tubes to separate from each other by its own elasticity thus making a very strong attachment between the screw extender and the screw head. An additional benefit is that the relative rotation between the screw head and the cylindrical body is avoided.

In a preferred embodiment a rod reduction instrument is located within the cylindrical body. Advantageously the rod reduction instrument is essentially made of a shaft with a threaded distal part which is the counterpart of the cylindrical body internal threaded part. So when it is rotated within the cylindrical body the shaft may move along the cylindrical body main axis.

In another embodiment a set screw driver is (also or alternatively) located within said cylindrical body. In this case also, the set screw driver may also essentially be made of a shaft with a threaded distal part.

Advantageously the set screw driver comprises a torque limiting mechanism.

In one embodiment this mechanism includes a breakable pin and a thread free rotatable shaft. The pin is laterally crossing the rotatable shaft and its ends are fixed within the threaded rotatable shaft. The threaded and the threaded free shafts are rotatably linked to each other but when a certain torque is reached the pin breaks and each shaft may freely rotates with respect to the other shaft.

In another embodiment a screw releasing instrument is (also or alternatively) located within said cylindrical body.

Advantageously the screw releasing instrument is essentially made of a shaft with a threaded distal part.

In a particularly interesting embodiment, the same shaft with a threaded distal part is used for the rod reduction instrument (and potential sponylolisthesis), the set screw driver and the screw releasing mechanism.

The tissue dilatation sleeve according to the invention comprises a flexible conical part which is adapted to be temporarily fixed to the distal part of an instrument such as a screw extender as defined in the previous claims.

In one embodiment the conical part is made of several longitudinal flexible blades having each a substantially triangular shape.

### DETAILED DESCRIPTION OF THE INVENTION

The invention will be better understood in the following part of this document, with non-limiting examples illustrated by the following figures:

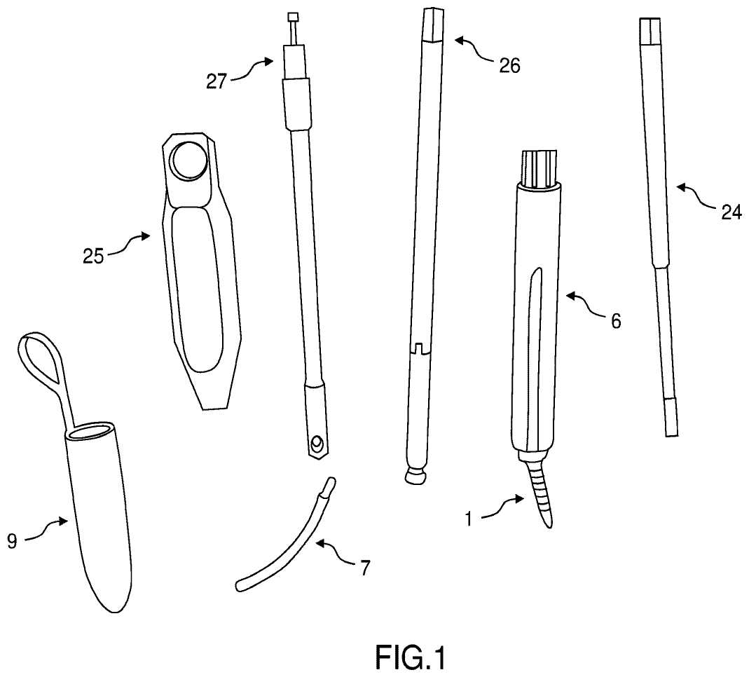

FIG. 1 shows an implant kit according to the invention

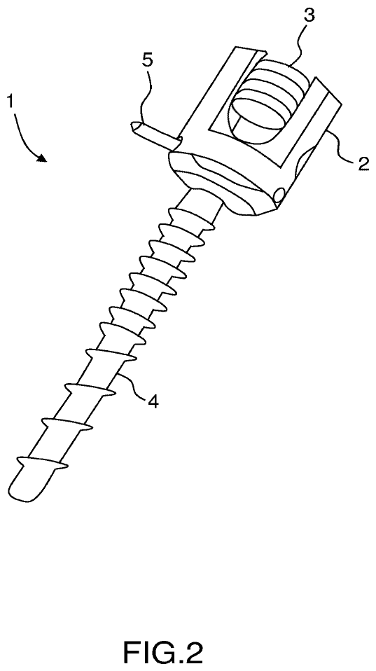

FIG. 2 shows an example of a lockable poly-axial pedicle screw according to the invention

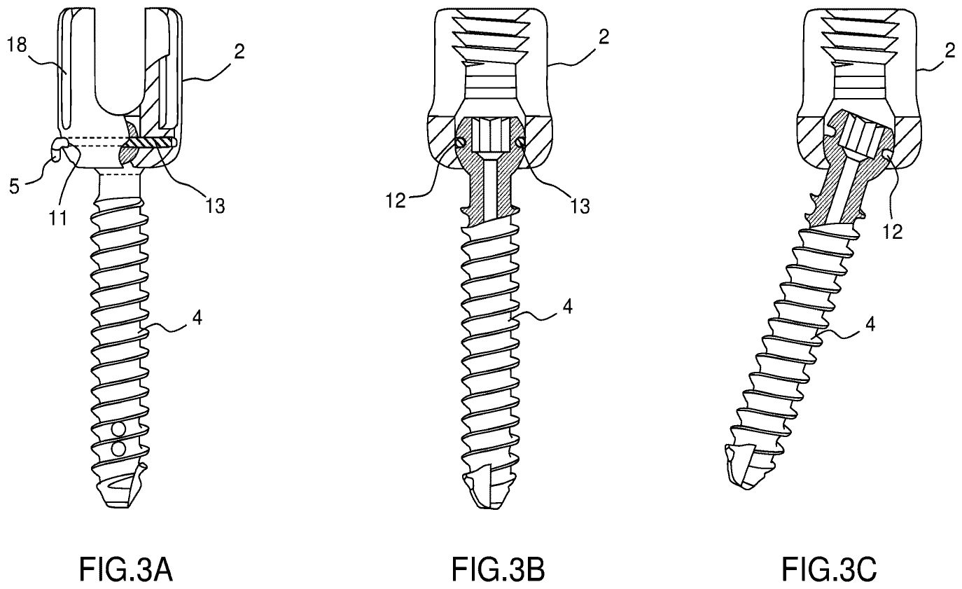

FIGS. 3A to 3C are cross-sections and partial cut views of the screw of FIG. 2

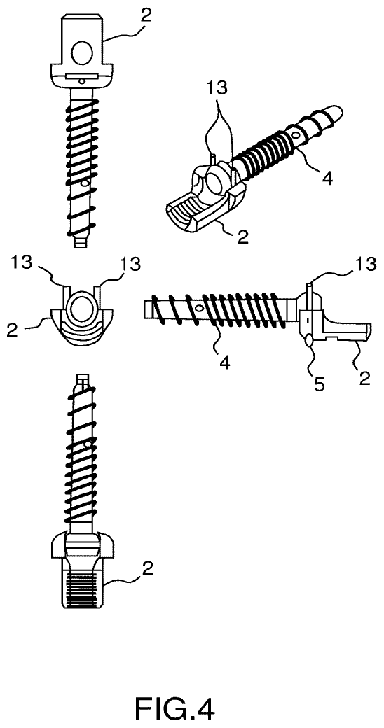

FIG. 4 represents different views (complete and partial) of the screw of FIG. 2

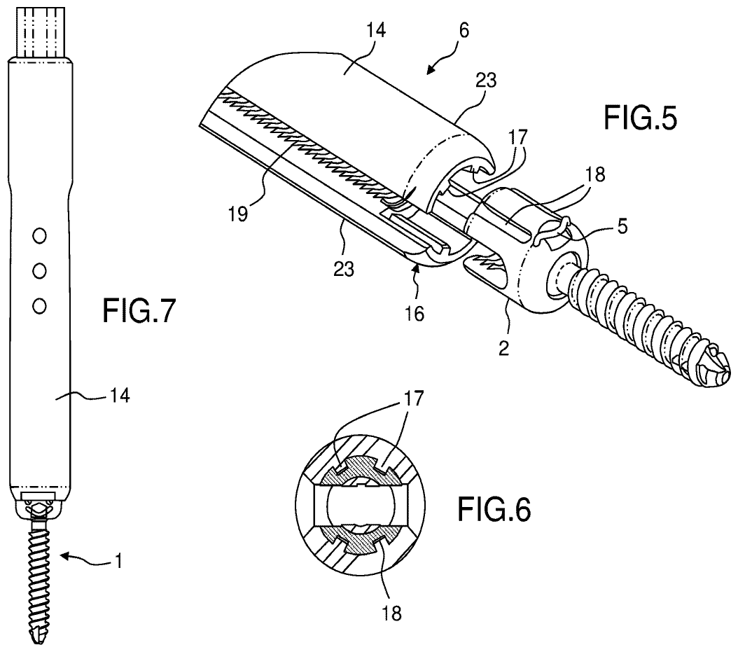

FIG. 5 shows the distal part of a screw extender according to the invention, together with the screw of FIG. 2

FIG. 6 is a cross section of the screw extender distal part

FIG. 7 is a global view of a screw extender with a screw

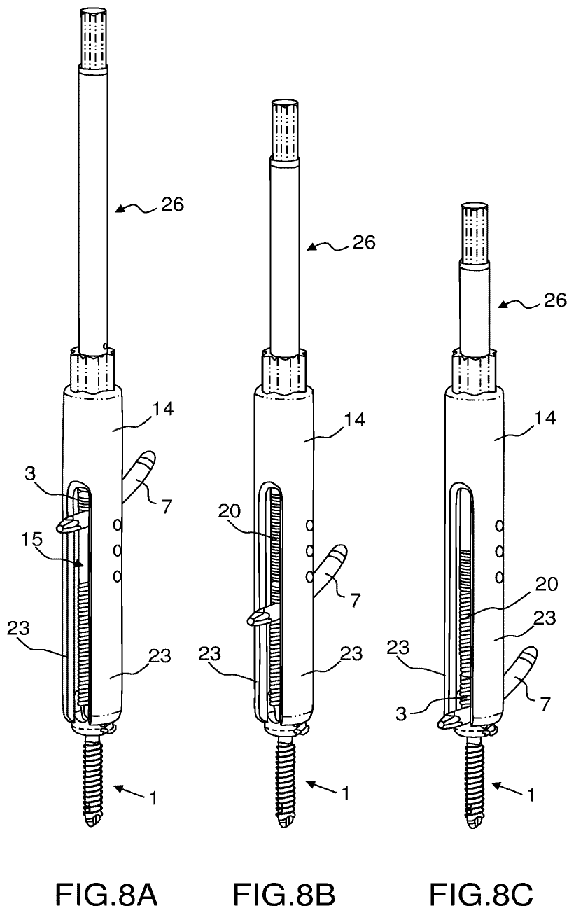

FIGS. 8A to 8C represent the positioning of the rod in the screw head, rod reduction and the tightening of the set screw

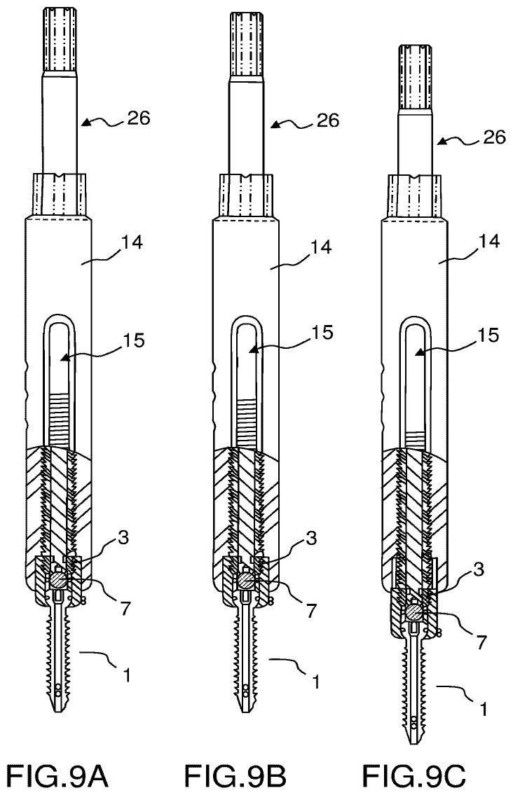

FIGS. 9A to 9C illustrate the release of the screw with respect to the screw extender

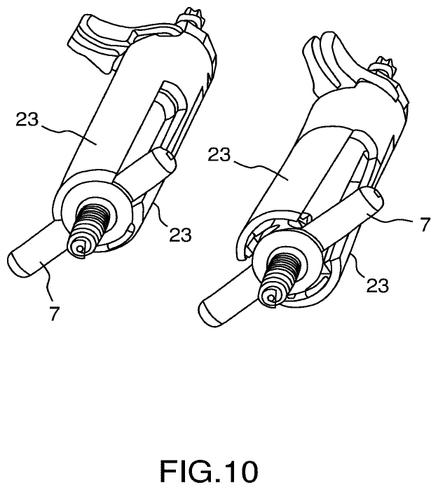

FIG. 10 shows a screw extender which includes a mechanism for laterally expanding the screw extender distal part

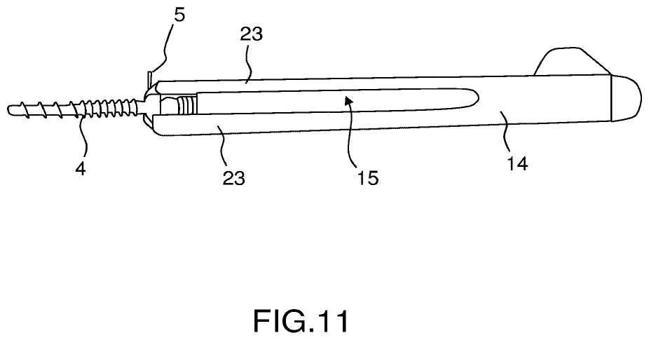

FIG. 11 is another representation of the screw extender of FIG. 10

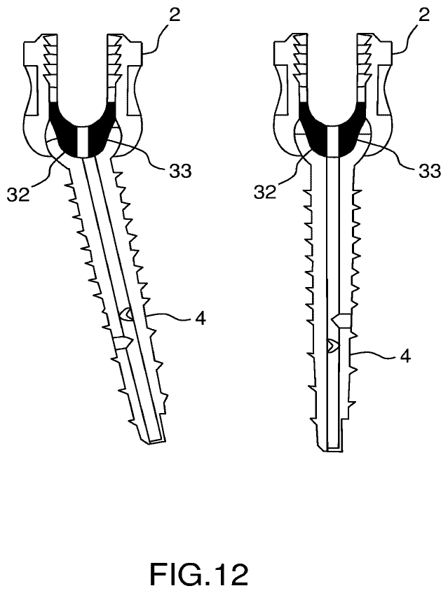

FIG. 12 shows another embodiment of a pedicle screw according to the invention

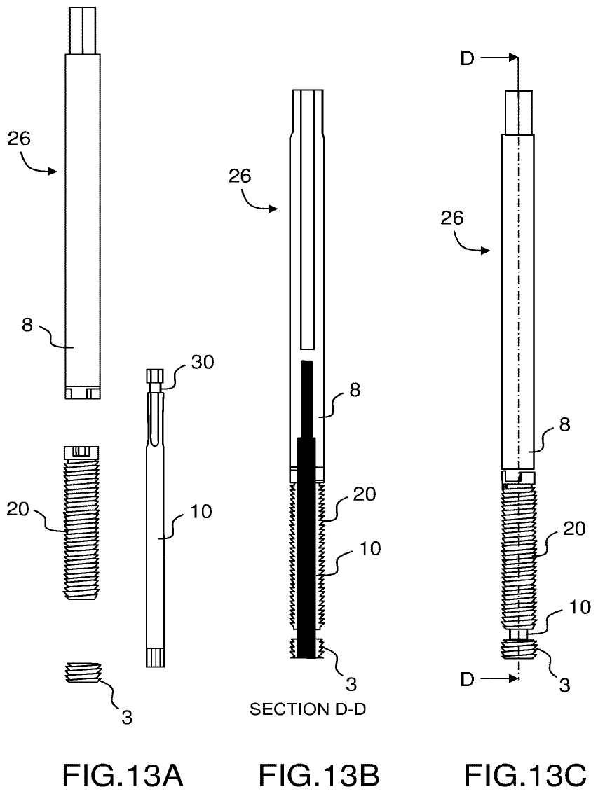

FIGS. 13A to 13C show different views of a rotatable shaft which is used in a rod reduction instrument, a set screw driver and a screw releasing instrument

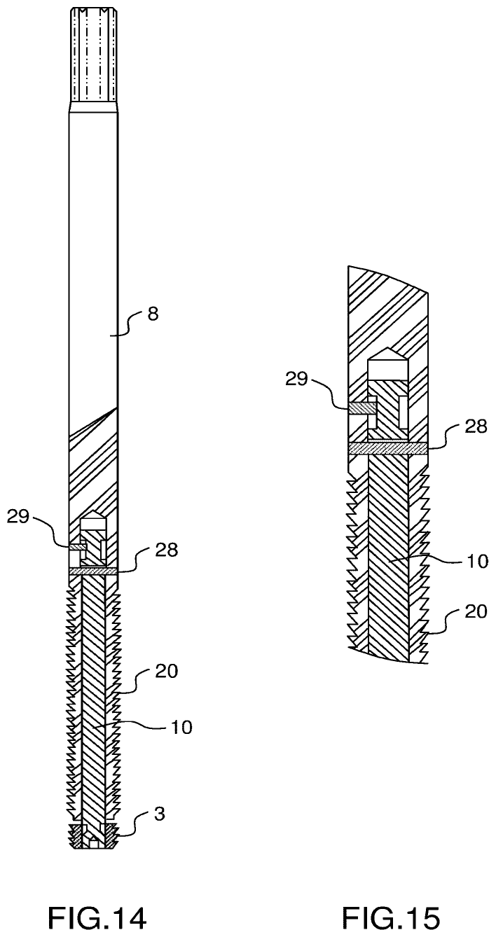

FIGS. 14 and 15 illustrate a torque limiting mechanism

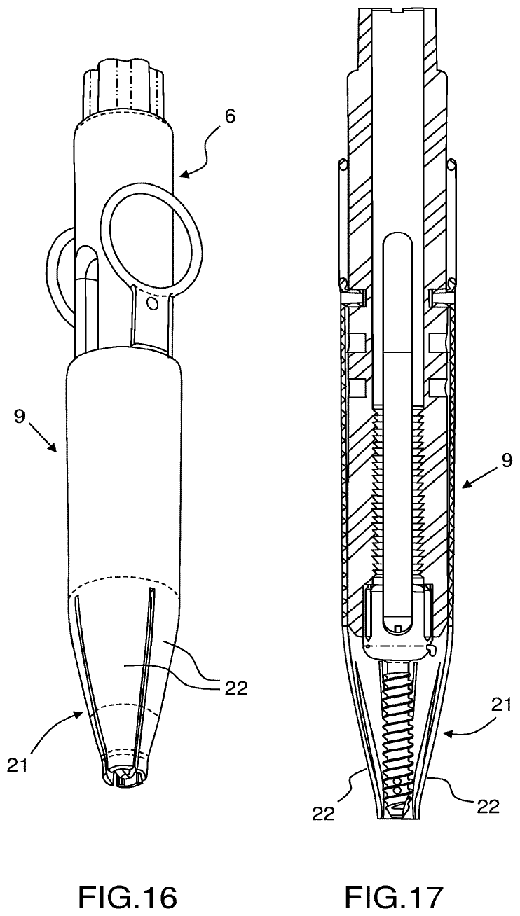

FIGS. 16 and 17 show the use of a tissue dilatation sleeve

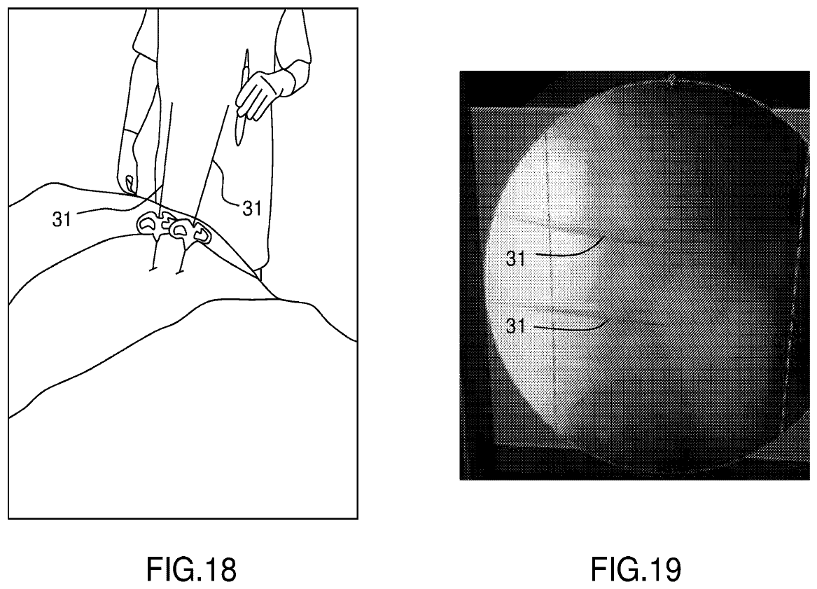

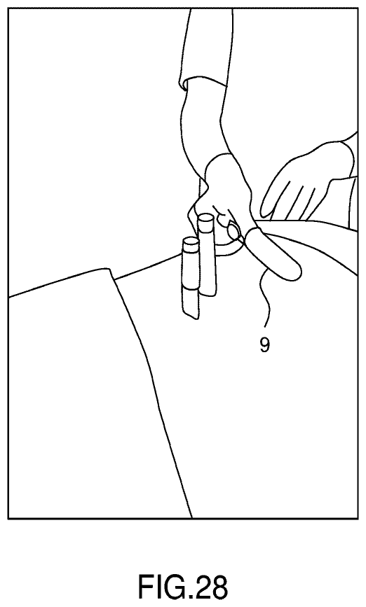

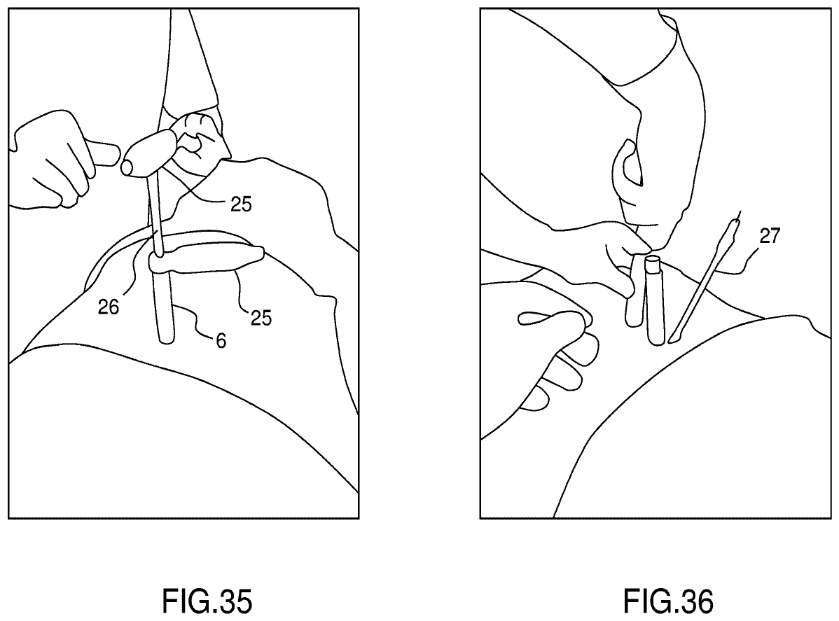

FIGS. 18 to 38 show a procedure using an implant kit according to the invention

### NUMERICAL REFERENCES USED IN THE FIGS

1. Pedicle screw
2. Head
3. Set screw
4. Threaded portion
5. Locking element
6. Screw extender
7. Rod
8. Multi-use instrument upper part
9. Tissue dilatation sleeve
10. Torque driver
11. Head passage
12. Threaded portion passage
13. Branch
14. Cylindrical body
15. Slot
16. Cylindrical body distal part
17. Ridge
18. Groove
19. Screw extender internal threaded part
20. Multi-use instrument lower part
21. Conical part
22. Blade
23. Half tube
24. Screw driver
25. Handle
26. Multi-use instrument (Rod reduction/Set screw driver/screw release)
27. Rod inserting instrument
28. Breakable pin
29. Lateral pin
30. Circular groove
31. Puncturing needle/Guide wire
32. Concave screw top
33. Convex upper half ball

The examples below more precisely relate to a thoracolumbar fusion system consisting of pedicle screws and rods combined with single use instruments. A typical pedicle screw system consists of the screw implants and the instruments for placing the screws.

FIG. 1 shows an example of an implant kit according to the invention.

This kit contains a tissue dilatation sleeve 9, a handle 25, a rod 7, a rod inserting instrument 27, a shaft 26 which can be used as a rod reduction instrument and/or a set screw driver and/or a screw releasing instrument, a pedicle screw 1, a screw extender 6 and a screw driver 24.

The lockable poly-axial screw 1 illustrated in particular in FIGS. 2 to 4 includes a head 2 and a threaded portion 4. FIG. 2 also shows a set screw 3 which may be fixed to the head after the insertion of a rod 7. The screw 1 furthermore comprises a locking element 5 having a U-shape. When the locking element 5 is fully inserted in the screw head 2 the orientation of threaded portion 4 is blocked with respect to the head 2. Inversely, when the locking element is retrieved, the threaded portion 4 can be freely oriented with respect to the screw head 2.

The lockable poly-axial screw according to the invention may therefore be transformed into a mono-axial screw, thus allowing having mono-axial and poly-axial capability in the same product. A blocking system defined previously allows the surgeon to choose if he/she wants to use the screw in mono-axial or poly-axial mode. As mentioned mono-axial capability is achieved by pushing the locking element (clip) 5 and poly-axial capability is achieved by removing the clip 5. The clip 5 is just an example of a blocking system; other technical solutions can also be imagined such as a pin.

Preferably, in the mono-axial mode the head may still freely rotate around its own axis, with respect to the threaded portion. Such a mechanism may be obtained with a U-shape clip and with an annular groove located around the upper part of the threaded portion. In this case the branches of the clip are sliding within the annular groove.

Any orientation of the axis can be considered when the mono-axial is used, i.e. the screw axis and the screw head may be oriented along different directions.

FIGS. 5 to 7 represent the attachment of a pedicle screw 1 to the distal end 16 of a screw extender 6, by inserting the screw head 2 within the distal end 16. In this operation the head 2 is guided with a plurality of ridges 17 located within the distal end 16 and grooves located on the head 2. With such a system the screw head is better retained within the screw extender 6. Any suitable material can be used for the screw extender 6 (plastic, polymer, metal, etc . . . ).

FIGS. 8A to 8C represent the positioning of the rod 7 in the screw head 2, a rod reduction and the tightening of the set screw 3 in the screw head 2.

The multi-use instrument 26 (see also FIGS. 13A to 13B) is defined by an upper part 8 and a lower (threaded) part 20.

The rod 7 may be pushed downwards by rotating the multi-use instrument 26 within the cylindrical body 14.

After the rod insertion within the head 2, the set screw 3 is fixed to the head 2 by further rotating the multi-use instrument 26.

The multi-use instrument 26 is also provided with a torque limiting mechanism (see FIGS. 14 and 15). When the set screw 3 is fixed within the head 2 and the multi-use instrument 26 further rotated, the torque increases, up to a point where the pin 28 breaks. A further rotation of the multi-use instrument 26 has therefore no more effect on the set screw 3. From that point the further rotation of the multi-use instrument 26 only induces a downwards pressure on the screw head 2. The screw 1 is therefore progressively separated from the screw extender (see FIGS. 9A to 9C).

This screw releasing mechanism from an instrument offers the possibility to release the screw 1 from the screw extender without laterally expanding the screw extender 6.

It should be mentioned at this stage that this mechanism is not limited to the release of pedicle screws. Any other item may be used.

To summarize, the same instrument 26 can be used for rod reduction, for fixing a set screw to a screw head and for releasing a screw from a screw extender.

It should be underlined that the invention is not limited to this triple use of the same instrument. A double use is also comprised, for instance rod-reduction and fixation of the screw set to the screw head.

FIGS. 10 and 11 show an alternative solution to attach a pedicle screw to an screw extender, by rotating an inside tube (not illustrated) the half-tubes 23 are expanded by their own elasticity. This allows a screw to be inserted and fixed to it by for example clamping the outer surface around the screw. The same principle can be used as an alternative to detach the screw extender from a screw.

The clamping system also achieves part of its rigidity, by resting on support surfaces on the screw head.

FIG. 12 shows a concave screw top 32, inside a convex upper half ball 33, allowing the rod 7 and the set screw 3 to be set lower in the screw head 2, thus decreasing the total build height, and increasing the strength and rigidity of the system.

FIGS. 16 and 17 represents a tissue dilatation sleeve 9 containing four triangular flexible blades 22 intervened into each other and forming a cone 21. The cone 21 is mounted at the tip of a screw extender 6, with a tear off spiral. This allows the tissue to be pushed aside as the screw extender 6 is inserted into a body. Once in place, the surgeon may remove the sleeve 9 while the screw extender remains in the body. Any suitable number of blades can be used for forming the cone.

FIGS. 18 to 38 show a procedure using the items which have been previously presented.

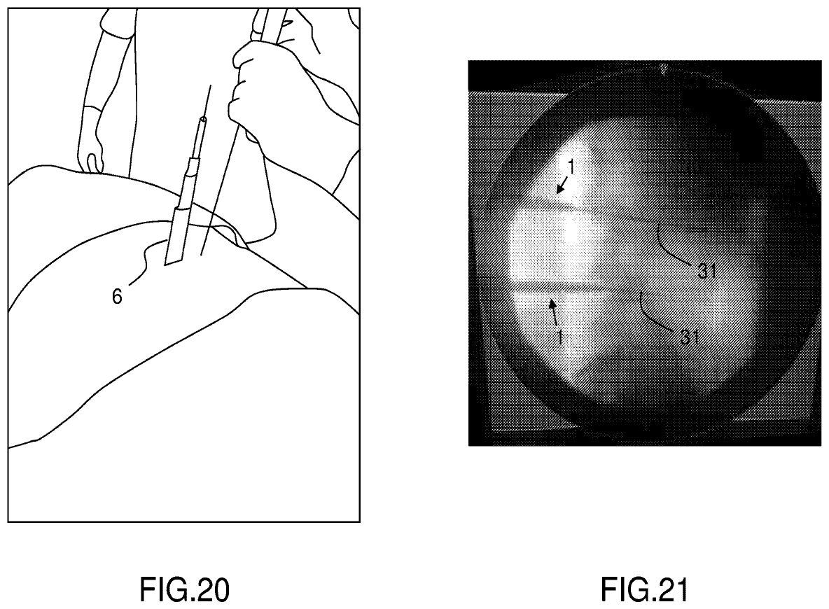

In a first step (FIGS. 18 and 19) two puncturing guide wires 31 are positioned in the spine. A first screw extender 6 with a screw 1 attached and surrounded by a dilatation sleeve is then inserted through the tissue (FIGS. 20 and 21), and along the guide wire 31. The screw extender 6 is rotated and/or pushed.

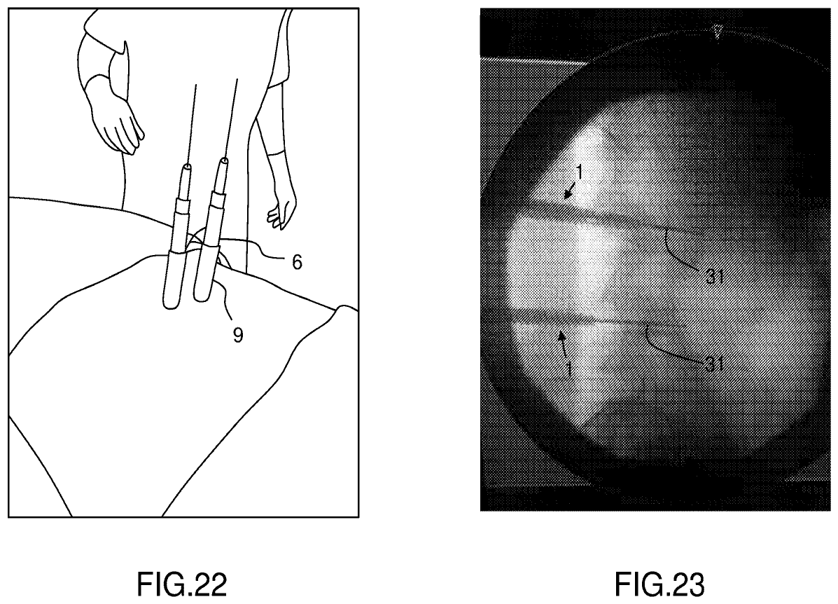

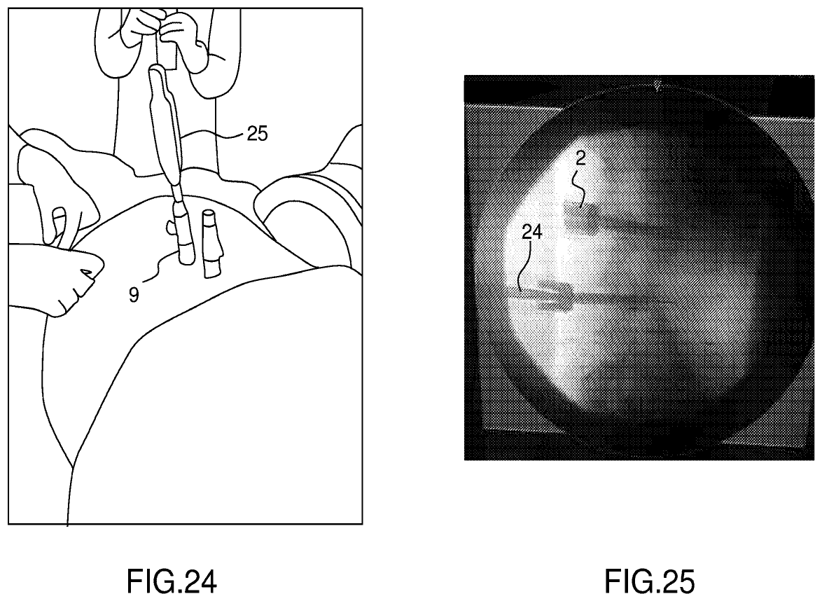

A similar operation is carried out with a second screw extender 6 and screw 1 (FIGS. 22 and 23). A screw driver 24 is inserted within the screw extender 6. Its distal end is introduced within the upper part of the screw threaded portion 4. The screws 1 are then rotated and enter the vertebrae (FIGS. 24 and 25).

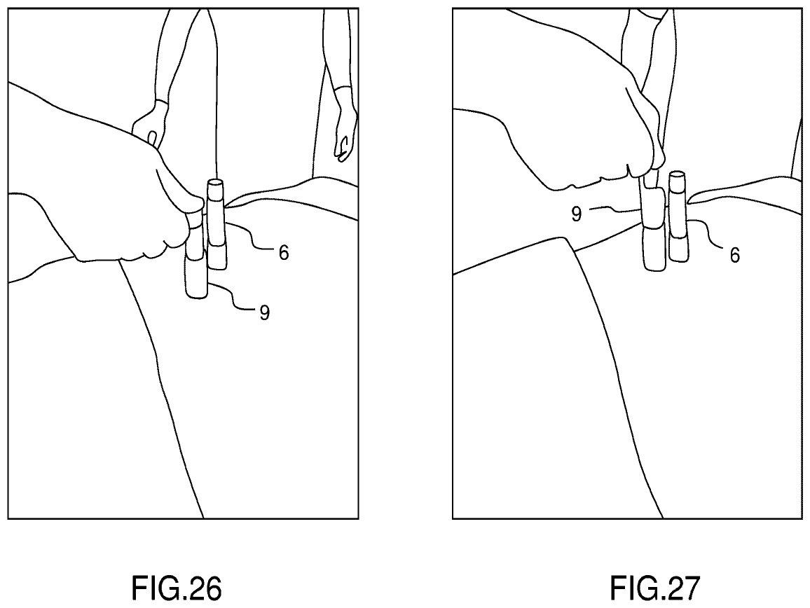

The tissue dilatation sleeves 9 are removed (FIGS. 26 to 28).

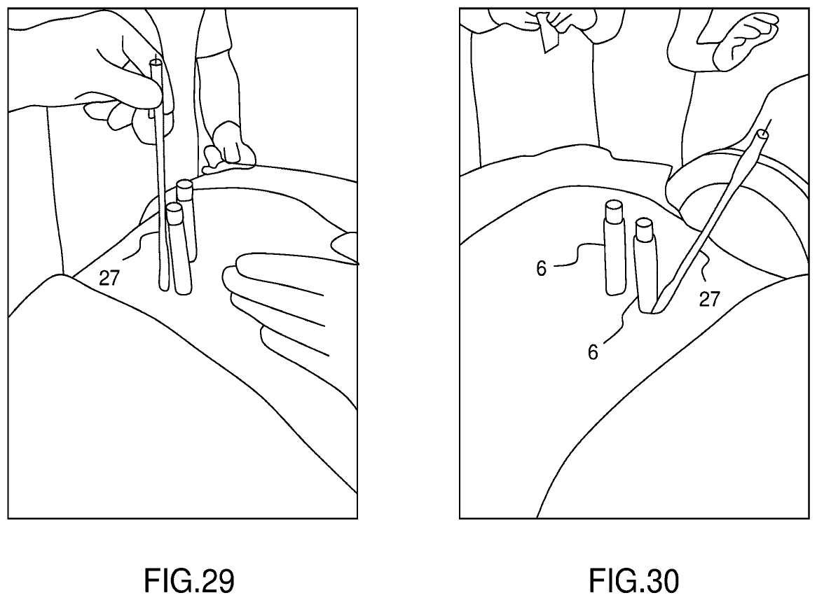

A rod inserting instrument 27 with a rod 7 at its end is transversally crossing the tissue (FIGS. 29 and 30).

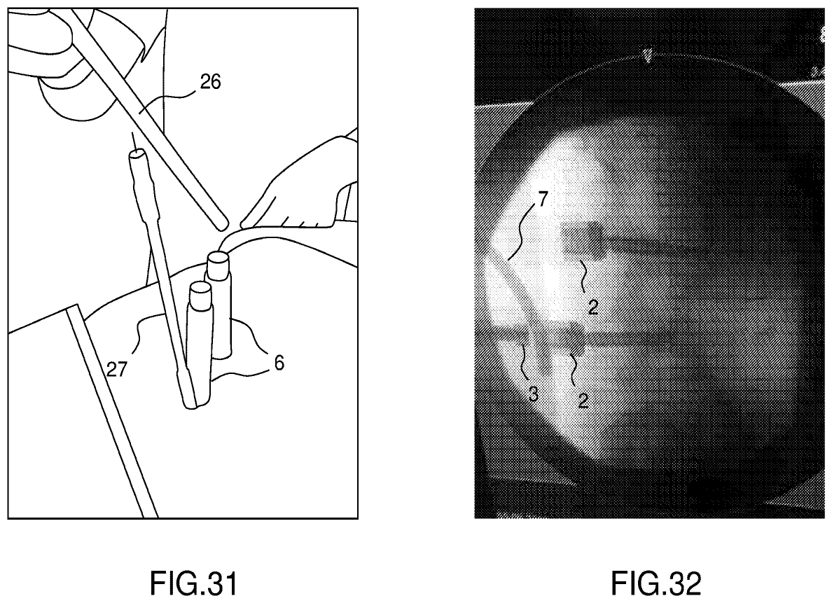

The rod 7 is positioned above the screw head 2 (FIG. 32) and the multi-use instrument 26 is introduced within the screw extender 6, to such an extent that the set screw 3 is positioned above the rod 7, in line with the screw head 2 (FIGS. 31 and 32).

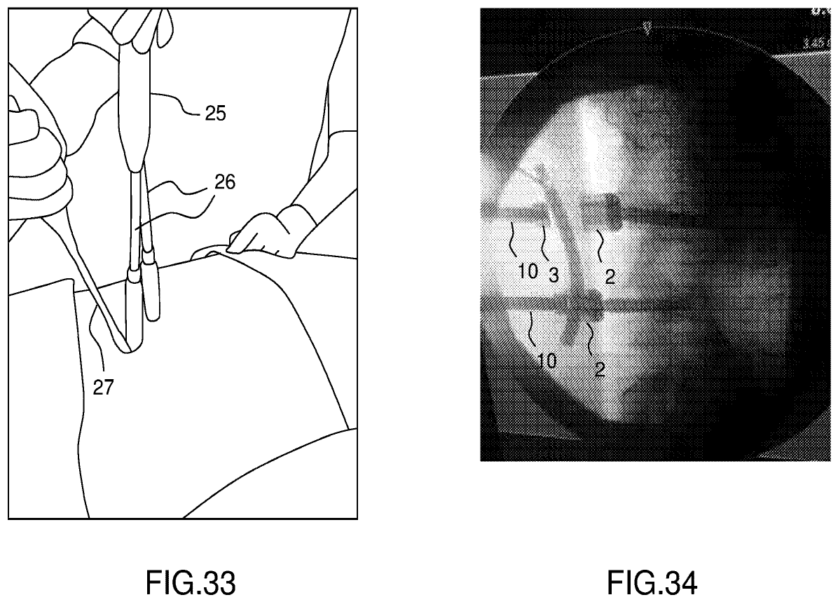

FIGS. 33 to 36 show the rod placement within the screw heads 2 and the fixation of the set screw 3 within the screw head 2.

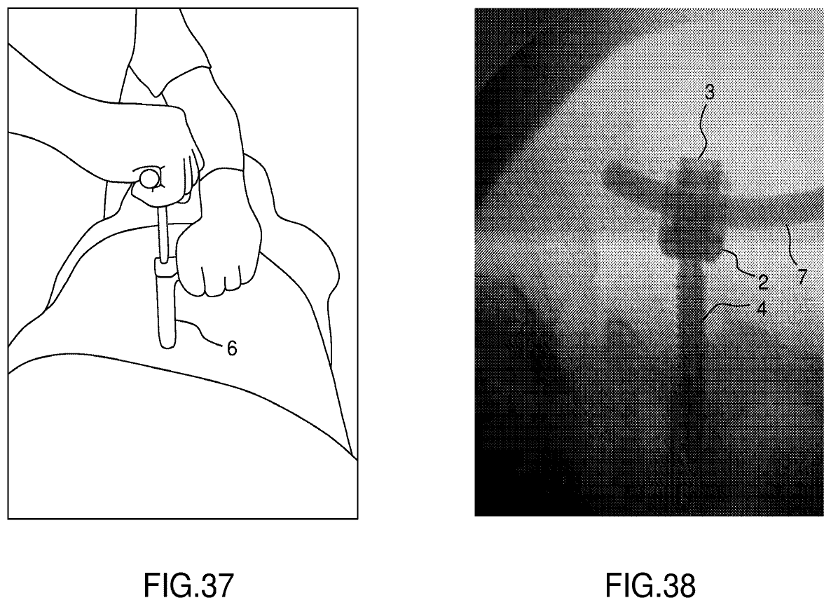

FIGS. 37 and 38 illustrate the screw release from the screw extender 6 and the screw 1, the rod 7 and the screw set 3 in their definitive location.

The invention is of course not limited to those illustrated examples.

The screw extender according to the invention may be used with mono-axial, poly-axial or lockable poly-axial screws.

1. A lockable poly-axial orthopedic screw comprising:
a head and a threaded portion, the head and the threaded portion forming two separate elements that are fixed to each other, the threaded portion having a spherical upper part engaging with a corresponding spherical surface of the head; and
a locking element that, in an unlocked position, is configured to allow an independent orientation of the threaded portion relative to the head, and in a locked position, is configured to transform the poly-axial screw into a mono-axial screw having a direction of orientation of a main axis of the threaded portion fixed relative to the head.

2. The screw according to claim 1, wherein the locking element includes:
an annular groove circumferentially surrounding the spherical upper part of the threaded portion; and
a clip configured to engage with a portion of the annular groove.

3. The screw according to claim 2, wherein, in the locked position, the clip is engaged with a portion of the annular groove of the spherical upper part, such that the independent orientation of the threaded portion relative to the head is blocked, but a rotation of the threaded portion relative to the head is possible.

4. The screw according to claim 2, wherein the locking element further includes:
an opening in the head for accommodating the clip, the opening reaching to the corresponding spherical surface,
wherein the annular groove of the threaded portion and the opening of the head are configured to be aligned by orientation of the threaded portion relative to the annular groove, for inserting the clip to the portion of the annular groove.

5. The screw according to claim 1, wherein the head includes:
a longitudinal groove or a longitudinal ridge, the longitudinal groove or longitudinal ridge being configured to receive a corresponding longitudinal ridge or a corresponding longitudinal groove, respectively, the corresponding longitudinal ridge or the corresponding longitudinal groove located within a distal end of a screw extender of the implant kit.

6. The screw according to claim 1, further comprising:
a concave seat located in a proximal end of the threaded portion and a corresponding convex engagement surface located at a distal part of the head.

7. The screw according to claim 1, wherein the locking element includes:
an annular groove circumferentially surrounding the spherical upper part of the threaded portion; and
a branch that is configured to engage with a portion of the annular groove.

8. The screw according to claim 7, wherein in the locked position, the branch engages with the portion of the annular groove to block the independent orientation between the threaded portion and the head, and in the unlocked position, the annular groove is unobstructed by the branch to permit the independent orientation between the threaded portion and the head.

9. The screw according to claim 1, wherein the head includes an engagement mechanism configured to engage with a screw extender by an insertion to the screw extender in a direction of a main axis of the head and the screw extender.

10. The screw according to claim 1, wherein in the locked position, the threaded portion is configured to rotate relative to the head around the main axis of the threaded portion.

11. An implant kit having the lockable poly-axial orthopedic screw according to claim 1.
